(19) United States
(12) Patent Application Publication
(10) Pub. No.: US 2003/0171304 A1
(43) Pub. Date: Sep. 11, 2003

(54) Pyridine-2-yl-aminoalkyl carbonyl glycyl-$g(b)-alanine and derivatives thereof

(76) Inventor: G?uuml;nter Hölzeman, (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/297,989

(22) PCT Filed: Jun. 12, 2001
(86) PCT No.: PCT/EP01/06661

(30) Foreign Application Priority Data
Jun. 13, 2000 (DE) 100-28-402.7

(51) Int. Cl. A61K 38/04; C07K 5/04; C 07D 4 1/02

(52) U.S. Cl. 514/19; 546/264; 546/159; 546/256; 546/280.4

(57) ABSTRACT

The invention relates to compounds of the formula I

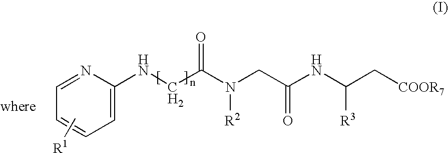

R¹ is H, A, Ar, Hal, —OH, —O—A, —CF₃ or —OCF₃;

R² and R⁷ are H or A;

R³ is

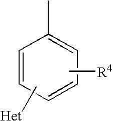

R⁴, R⁵ and R⁶ are in each case, independently of each other, H, A, Hal, —OH, —O—A, —CF₃, —OCF₃, —CN, —NH₂, —A—NH₂;

A is C₁-C₆-alkyl;

Ar is a substituent which is formed by an aromatic radical which is optionally substituted once, twice or three times by R⁵ and which has from 1 to 3 ring structures which are optionally fused with other ring structures to form a fused ring system;

Het is a substituent which is formed by a heterocycle which has from 1 to 3 ring structures, with each ring structure being saturated, unsaturated or aromatic and being optionally fused with other ring structures to form a fused ring system, and the heterocycle possessing a total of from 1 to 4 N, O and/or S atoms in the ring structures and being optionally substituted by R⁶;

Hal is F, Cl, Br or I;

n is 2, 3, 4, 5 or 6

and to their use.

The invention relates to compounds of the formula I

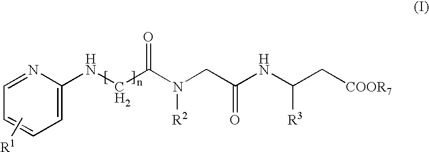

where

R¹ is H, A, Ar, Hal, —OH, —O—A, —CF₃ or —OCF₃;

R² and R⁷ are H or A;

R³ is

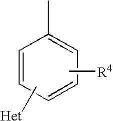

R⁴ and R⁶ are in each case, independently of each other, H, A, Hal, —OH, —O—A, —CF₃, —OCF₃, —CN, —NH₂, —A—NH₂;

R⁵ is in each case, independently of each other, H, A, Hal, —OH, —O—A, —CF₃, —OCF₃, CN, NO₂;

A is C₁-C₆-alkyl;

Ar is a substituent which is formed by an aromatic radical which is optionally substituted once, twice or three times by R⁵ and which has from 1 to 3 ring structures which are optionally fused with other ring structures to form a fused ring system;

Het is a substituent which is formed by a heterocycle which has from 1 to 3 ring structures, with each ring structure being saturated, unsaturated or aromatic and being optionally fused with other ring structures to form a fused ring system, and the heterocycle possessing a total of from 1 to 4 N, O and/or S atoms in the ring structures and being optionally substituted by R⁶;

Hal is F, Cl, Br or I;

n is 2, 3, 4, 5 or 6

and the well-tolerated salts and solvates thereof.

WO 97/26250 and WO 97/24124 deal with compounds of a related substance class.

WO 97/26250 relates to compounds of the general formula

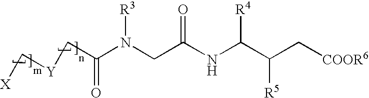

and their use as integrin inhibitors, where X, Y, m, n, R³, R⁴, R⁵ and R⁶ have the meanings given in WO 97/26250. X is a 5-membered to 6-membered monocyclic aromatic ring which has form 0 to 4 nitrogen, oxygen or sulfur atoms and which is optionally substituted by R¹ or R², or is a 9-membered to 10-membered polycyclic ring system in which at least one ring is aromatic and which possesses from 0 to 4 nitrogen, oxygen or sulfur atoms and which is optionally substituted. n and m are natural numbers from 0 to 6.

WO 97/24124 discloses vitronectin receptor antagonists of the formula

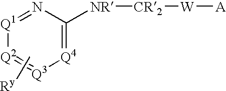

where the substituents have the meanings given in WO 97/24124.

The invention was based on the object of discovering novel compounds which possess valuable properties, in particular those which are used for producing drugs.

It has been found that, while being well tolerated, the compounds of the formula I, and their salts, possess very valuable pharmacological properties. Above all, they act as integrin inhibitors, in connection with which they inhibit, in particular, the interactions of the αvβ3 or αvβ5 integrin receptors with ligands, such as, for example, the binding of vitronectin to the αvβ3 integrin receptor. Integrins are membrane-bound, heterodimeric glycoproteins which are composed of an α-subunit and a smaller β-subunit. The relative affinity and specificity for binding a ligand is determined by the combination of the different α- and β-subunits. The compounds according to the invention exhibit a particularly high degree of activity in the case of the integrins αvβ1, αvβ3, αvβ5, αllbβ3 and also αvβ6 and αvβ8, preferably in the case of αvβ3, αvβ5 and αvβ6. Potent selective inhibitors of the αvβ³ integrin have been found, in particular. The αvβ3 integrin is expressed on a number of cells, e.g. endothelial cells, cells of the smooth musculature of the blood vessels, for example of the aorta, cells for breaking down the bone matrix (osteoclasts) and tumor cells.

The effect of the compounds according to the invention can be demonstrated, for example, using the method which is described by J. W. Smith et al. in J. Biol. Chem. 1990, 265, 12267-12271.

In Science 1994, 264, 569-571, P. C. Brooks, R. A. Clark and D. A. Cheresh describe how the origin of angiogenesis depends on the interaction between vascular integrins and extracellular matrix proteins.

In Cell 1994, 79, 1157-1164, P. C. Brooks, A. M. Montgomery, M. Rosenfeld, R. A. Reisfeld, T. Hu, G. Klier and D. A. Cheresh describe the possibility of using a cyclic peptide to inhibit this interaction and thereby initiate the apoptosis (programmed cell death) of angiogenic vascular cells. This paper described, for example, αvβ3 antagonists or antibodies against αvβ3 which shrink tumors by initiating apoptosis.

The experimental proof that the compounds according to the invention also prevent living cells from adhering to the corresponding matrix proteins, and accordingly also prevent tumor cells from adhering to matrix proteins, can be provided in a cell adhesion test in analogy with the method of F. Mitjans et al., J. Cell Science 1995, 108, 2825-2838.

The compounds of the formula I are able to inhibit the binding of metalloproteinases to integrins and in this way prevent the cells from being able to use the enzymic activity of the proteinase. An example can be found in the ability of a cyclo-RGD peptide to inhibit the binding of MMP-2 (matrix metalloproteinase 2) to the vitronectin receptor αvβ3, as described in P. C. Brooks et al., Cell 1996, 85, 683-693.

Compounds of the formula I which block the interaction of integrin receptors and ligands, such as the binding of fibrinogen to the fibrinogen receptor (glycoprotein IIb/IIIa), prevent, as antagonists, the spread of tumor cells by metastasis and can therefore be employed as substances having an antimetastatic effect in operations in which tumors are removed surgically. This is substantiated by the following observations:

Tumor cells spread from a local tumor into the vascular system as a result of microaggregates (microthrombi) being formed by the tumor cells interacting with blood platelets. The tumor cells are shielded by the protection afforded in the microaggregate and are not recognized by the cells of the immune system. The microaggregates are able to settle on vessel walls, thereby facilitating further penetration of tumor cells into the tissue. Since formation of the microthrombi is mediated by the binding of ligands to the corresponding integrin receptors, e.g. αvβ3 or αllbβ3, on activated blood platelets, the corresponding antagonists can be regarded as being effective inhibitors of metastasis.

The effect of a compound on an αvβ5 integrin receptor, and consequently its activity as an inhibitor, can be demonstrated, for example, using the method which is described by J. W. Smith et al. in J. Biol. Chem. 1990, 265, 12267-12271.

The compounds of the formula I can be employed as drug active compounds in human and veterinary medicine, in particular for the prophylaxis and/or therapy of circulatory diseases, thrombosis, cardiac infarction, arteriosclerosis, stroke, angina pectoris, tumor diseases, such as the development or metastasis of tumors, osteolytic diseases, such as osteoporosis, pathologically angiogenic diseases, such as inflammations, opththalmological diseases, diabetic retinopathy, macular degeneration, myopia, ocular histoplasmosis, rheumatoid arthritis, osteoarthritis, rubeotic glaucoma, ulcerative colitis, Crohn's disease, atherosclerosis, psoriasis restenosis following angioplasty, multiple sclerosis, viral infection, bacterial infection and fungal infection, and in association with acute kidney failure and in association with wound healing, for the purpose of promoting the healing process.

αᵥβ₆ is a relatively rare integrin (Busk et al., 1992 J. Biol. Chem. 267(9), 5790) which is formed in increased quantity in association with repair processes in epithelial tissue and which preferentially binds the natural matrix molecules fibronectin and tenascin (Wang et al., 1996, Am. J. Respir. Cell Mol. Biol 15(5), 664). The physiological and pathological functions of αᵥβ₆ are still not known with precision: however, it is suspected that this integrin plays an important role in physiological processes and diseases (e.g. inflammations, wound healing and tumors) in which epithelial cells are involved. Thus, αᵥβ₆ is expressed on keratinocytes in wounds (Haapasalmi et al., 1996, J. Invest. Dermatol. 106(1), 42), from which it can be assumed that it is possible for agonists or antagonists of said integrin to influence other pathological events in the skin, such as psoriasis, in addition to wound healing processes and inflammations. Furthermore, αᵥβ₆ plays a role in the respiratory tract epithelium (Weinacker et al., 1995, Am. J. Respir. Cell Mol. Biol. 12(5), 547), which means that it ought to be possible to use corresponding agonists/antagonists of this integrin for successfully treating respiratory tract diseases such as bronchitis, asthma, lung fibroses and respiratory tract tumors. Finally, it is known that αᵥβ₆ also plays a role in the intestinal epithelium, which means that corresponding integrin agonists/antagonists ought to be of use in treating inflammations, tumors and wounds of the stomach/intestinal tract.

The effect of a compound on an αvβ6 integrin receptor, and consequently its activity as an inhibitor, can be demonstrated, for example, using the method which is described by J. W. Smith et al. in J. Biol. Chem. 1990, 265, 12267-12271.

The compounds of formula I can be employed as substances having an antimicrobial effect in operations in which biomaterials, implants, catheters or heart pacemakers are used.

In this context, their effect is that of an antiseptic. The efficacy of the antimicrobial activity can be demonstrated using the method described by P. Valentin-Weigund et al. in Infection and Immunity, 1988, 2851-2855.

A measure of the uptake of a drug active compound into an organism is its bioavailability.

If the drug active compound is administered to the organism intravenously in the form of an injection solution, its absolute bioavailability, i.e. the proportion of the drug which reaches the systemic blood, i.e. the general circulation, in unaltered form is 100%.

When a therapeutic active compound is administered orally, the active compound is as a rule present in the formulation as a solid and has, therefore, first of all to be dissolved so that it can overcome the entry barriers, for example the gastrointestinal tract, the oral mucosa, the nasal membranes or the skin, in particular the stratum corneum, or can be absorbed by the body. Data on pharmacokinetics, i.e. on bioavailability, can be obtained in analogy with the method described by J. Shaffer et al., J. Pharm. Sciences, 1999, 88, 313-318.

The compounds of the formula I possess at least one chiral center and can therefore occur in several stereoisomeric forms. All these forms (e.g. D and L forms) and their mixtures (e.g. the DL forms) are included in the formula.

The compounds according to the invention also include what are known as prodrug derivatives. Examples of these are compounds of the formula I which have been modified with alkyl or acyl groups, sugars or oligopeptides and which are rapidly cleaved, in the organism, to form the active compounds according to the invention. If the pharmacokinetic differences, which are frequently marginal, are ignored, the effect of the prodrug derivatives is equivalent to that of their active breakdown products, for which reason protection is sought for these compounds as well.

Furthermore, free amino groups or free hydroxyl groups which are substituents of compounds of the formula I may be provided with appropriate protective groups.

Solvates of the compounds of the formula I are understood as being additions of inert solvent molecules to the compounds of the formula I, which additions are formed due to the mutual attraction of the compounds and solvent molecules. Examples of solvates are monohydrates or dihydrates or addition compounds with alcohols, for example with methanol or ethanol.

In amplification of the abovementioned definitions, the meanings and preferred meanings of the substituents R¹, R², R³, R⁴, R⁵, R⁶, R⁷, A, Ar, Het, Hal and n are explained in detail below.

R¹ is preferably H, A, Hal or —OH, but in particular a methyl radical. R¹ is preferably in the para position to the pyridine nitrogen.

R² and R⁷ are preferably hydrogen.

R³ is preferably a phenyl radical which is substituted in the para position by Het and optionally at another by R⁴:

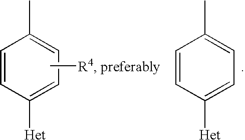

R⁴ is preferably H, A or Hal, but in particular hydrogen.

R⁵ is preferably methyl, ethyl, —OCH₃, —CF₃, OH, fluorine, chlorine or bromine.

A is linear or branched and has from 1 to 6, preferably 1, 2, 3, 4, 5 or 6, C atoms. A is preferably methyl, and, in addition, ethyl, n-propyl, isopropyl, n-butyl, sec-butyl or tert-butyl, and, in addition, also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, or 1,1,2- or 1,2,2-trimethylpropyl.

Methyl, ethyl, isopropyl, n-propyl, n-butyl or tert-butyl are particularly preferred for A.

Ar is a substituent which is formed by an aromatic radical which is optionally substituted once, twice or three times by R5 and which has from 1 to 3 ring structures which are optionally fused with other ring structures to form a fused ring system. The number of ring structures in an aromatic radical is identical to the number of ring openings which theoretically have to be performed in order to convert the aromatic radical into an acyclic compound. Ar preferably has only one ring structure.

Ar is preferably a phenyl, naphthyl, anthryl or biphenylyl radical which is optionally substituted once, twice or three times by R⁵, in particular a phenyl or naphthyl radical which is optionally substituted once, twice or three times. Ar is therefore preferably phenyl, o-, m- or p-methylphenyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-methoxyphenyl, o-, m- or p-ethoxyphenyl, o-, m- or p-trifluoromethylphenyl, o-, m- or p-fluorophenyl, o-, m- or p-chlorophenyl or o-, m- or p-bromophenyl, with 2,3-, 2,4-, 2,5-, 2,6-, 3,4- or 3,5-dimethylphenyl, 2,3-, 2,4-, 2,5-, 2,6-, 3,4- or 3,5-dihydroxyphenyl, 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,3-, 2,4-, 2,5-, 2,6-, 3,4- or 3,5-dimethoxyphenyl or 3-chloro-4-fluorophenyl, 4-fluoro-2-hydroxyphenyl, naphtalen-1-yl, naphthalen-2-yl or 2-, 3-, 4-, 5-, 6-, 7-or 8-methylnaphthalen-1-yl, 2-, 3-, 4-, 5-, 6-, 7- or 8-ethylnaphthalen-1-yl, 2-, 3-, 4-, 5-, 6-, 7- or 8-chloronaphthalen-1-yl, 2-, 3-, 4-, 5-, 6-, 7- or 8-fluoronaphthalen-1-yl, 2-, 3-, 4-, 5-, 6-, 7- or 8-bromonaphthalen-1-yl, 2-, 3-, 4-, 5-, 6-, 7- or 8-hydroxynaphthalen-1-yl, 1-, 3-, 4-, 5-, 6-, 7- or 8-methylnaphthalen-2-yl, 1-, 3-, 4-, 5-, 6-, 7- or 8-ethylnaphthalen-2-yl, 1-, 3-, 4-, 5-, 6-, 7- or 8-chloronaphthalen-2-yl, 1-, 3-, 4-, 5-, 6-, 7- or 8-fluoronaphthalen-2-yl, 1-, 3-, 4-, 5-, 6-, 7-, or 8-bromonaphthalen-2-yl, 1-, 3-, 4-, 5-, 6-, 7- or 8-hydroxynaphthalen-2-yl also being preferred. Very particular preference is given to Ar being phenyl, o-, m- or p-fluorophenyl, m- or p-chlorophenyl, p-methylphenyl, p-trifluoromethylphenyl, 3-chloro-4-fluorophenyl, 4-fluoro-2-hydroxyphenyl, naphthalen-1-yl or naphthalen-2-yl.

Het is a substituent which is formed by an optionally substituted heterocycle having from 1 to 3 ring structures; preference is given to the heterocycle having precisely one ring structure. The number of ring structures in a heterocycle is identical to the number of ring openings which theoretically have to be performed in order to convert the heterocycle into an acyclic compound. Insofar as is chemically possible, the ring structures may be saturated, unsaturated or aromatic independently of each other. A ring structure can be optionally fused with other ring structures to form a fused ring system. It is also possible for nonaromatic saturated or unsaturated ring structures to be linked to each other in analogy with fused ring systems, that is to share bonds with each other as is the case, for example, with steroids or with chroman. The heterocycle comprises a total of from 1 to 4 nitrogen, oxygen and/or S atoms, which replace the carbon atoms in the ring structures. These N, O and/or S atoms are preferably not adjacent. The heterocycle is optionally substituted by R⁶. Het is preferably pyridyl, quinolyl, thienyl, benzo[b]thienyl, indolyl, in particular pyridin-3-yl or pyridin-4-yl, quinolin-8-yl, thiophen-3-yl, benzo[b]thiophen-6-yl or indol-7-yl.

Hal is F, Cl, bromine or iodine, in particular F, Cl or bromine.

N is 2, 3, 4, 5 or 6, particularly preferably 3, 4 or 5, in particular, however, 3.

Compounds of the formula I in which R⁷ is not hydrogen, and also their solvates, are what is know as prodrugs, i.e. they are inactive in in-vitro experiments since they mask the biologically active carboxyl group. However, prodrugs are metabolically converted in the body into the biologically active form. The corresponding free acid, which corresponds to a compound of the formula I in which R⁷=H, and also its salts and solvates, is active in vitro.

Accordingly, the invention relates, in particular, to those compounds of the formula I in which at least one of said radicals has one of the abovementioned preferred meanings.

The invention also relates to a process for preparing compounds of the formula I, and also their salts and solvates, which process comprises the reaction

(a) of a compound of the formula II

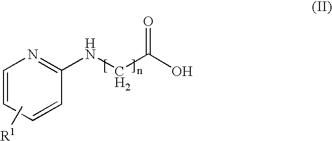

where R¹ and n have the abovementioned meanings, with a compound of the formula III

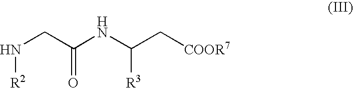

where R², R³ and R⁷ have the abovementioned meanings, with the radical R⁷≠H optionally being converted into the radical R⁷=H,

or comprises the reaction

(b) of a compound of the formula IV

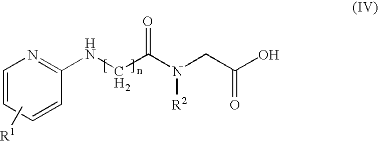

where R¹, R² and n have the abovementioned meanings, with a compound of the formula V

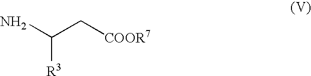

where R³ and R⁷ have the abovementioned meanings, and the radical R⁷≠H is optionally converted into the radical R⁷=H, or

(c) encompasses the conversion of one or more radicals R¹, R², R³, R⁴, R⁵, R⁶ and/or R⁷ of a compound of the formula I into one or more radicals R¹, R², R³, R⁴, R⁵, R⁶ and/or R⁷, by

i) alkylating a hydroxyl group and/or

ii) hydrolyzing an ester group to form a carboxyl group and/or

iii) esterifying a carboxyl group and/or

iv) alkylating an amino group and/or

v) acylating an amino group and/or

vi) converting a basic or acidic compound of the formula I into one of its salts or solvates by treating it with an acid or base.

The compounds of the formula I, and also the starting compounds for preparing them, are otherwise prepared in accordance with methods which are known per se and are described in the literature (e.g. in the standard works such as Houben-Weyl, Methoden der organischen Chemie [Methods of organic chemistry], Georg-Thieme-Verlag, Stuttgart), specifically under reaction conditions which are known and suitable for said reactions. In this connection, it is also possible to use variants which are known per se but which are not mentioned here in more detail.

If desired, the starting compounds can also be formed in situ, such that they are not isolated from the reaction mixture but instead immediately subjected to further reaction to form the compounds of the formula I.

Several—identical or different—protected amino groups and/or hydroxyl groups can also be present in the molecule of the starting compound. If the protective groups which are present are different from each other, they can in many cases be eliminated selectively (cf., in this regard: T. W. Greene, P. G. M. Wuts, Protective Groups in Organic Chemistry, 2nd ed., Wiley, New York 1991 or P. J. Kocienski, Protecting Groups, 1st ed., Georg Thieme Verlag, Stuttgart—New York, 1994, H. Kunz, H. Waldmann in Comprehensive Organic Synthesis, Vol. 6 (ed., B. M. Trost, I. Fleming, E. Winterfeldt), Pergamon, Oxford, 1991, pp. 631-701).

The expression “amino protecting group” is well known and refers to groups which are suitable for protecting (blocking) an amino group from chemical reactions. In particular, unsubstituted or substituted acyl, aryl, aralkoxymethyl or aralkyl groups are typical groups of this nature. Since the amino protecting groups are removed after the desired reaction (or reaction sequence), their nature and size is otherwise not critical; however, those with 1-20, in particular 1-8 C atoms are preferred. In connection with the present process, the expression “acyl group” is to be interpreted in the widest possible sense. It encompasses acyl groups which are derived from aliphatic, araliphatic, alicyclic, aromatic or heterocyclic carboxylic acids or sulfonic acids, and also, in particular, alkoxycarbonyl, alkenyloxycarbonyl, aryloxycarbonyl and, in particular, aralkoxycarbonyl groups. Examples of such acyl groups are alkanoyl, such as acetyl, propionyl or butyryl; aralkanoyl, such as phenylacetyl; aroyl, such as benzoyl or toluyl; aryloxyalkanoyl, such as phenoxyacetyl; alkoxycarbonyl, such as methoxycarbonyl, ethoxycarbonyl, 2,2,2-trichloroethoxycarbonyl, BOC or 2-iodoethoxycarbonyl; alkenyloxycarbonyl, such as allyloxycarbonyl (Aloc), aralkyloxycarbonyl, such as CBZ (synonymous with Z), 4-methoxybenzyloxycarbonyl (MOZ), 4-nitro-benzyloxycarbonyl or 9-fluorenylmethoxycarbonyl (Fmoc); 2-(phenylsulfonyl)ethoxycarbonyl; trimethylsilylethoxycarbonyl (Teoc) or arylsulfonyl, such as 4-methoxy-2,3,6-trimethylphenylsulfonyl (Mtr). Preferred amino protecting groups are BOC, Fmoc and Aloc, and, in addition, CBZ, benzyl and acetyl.

The expression “hydroxyl protecting group” is likewise well known and refers to groups which are suitable for protecting a hydroxyl group from chemical reactions. The abovementioned unsubstituted or substituted aryl, aralkyl, aroyl or acyl groups are typical of such groups, as are also alkyl groups, aryl groups or aralkylsilyl groups, or O,O- or O,S-acetals. The nature and size of the hydroxyl protecting groups is not critical since they are removed once again after the desired chemical reaction or reaction sequence; groups having 1-20, in particular 1-10, C atoms are preferred. Some examples of hydroxyl protecting groups are aralkyl groups, such as benzyl, 4-methoxybenzyl or 2,4-dimethoxybenzyl, aroyl groups, such as benzoyl or p-nitrobenzoyl, acyl groups, such as acetyl or pivaloyl, p-toluenesulfonyl, alkyl groups, such as methyl or tert-butyl and also allyl, alkylsilyl groups, such as trimethylsilyl (TMS), triisopropylsilyl (TIPS), tert-butyldimethylsilyl (TBS) or triethylsilyl, trimethylsilylethyl, aralkylsilyl groups, such as tert-butyldiphenylsilyl (TBDPS), cyclic acetals, such as isopropylidene acetal, cyclopentylidene acetal, cyclohexylidene acetal, benzylidene acetal, p-methoxybenzylidene acetal or o,p-dimethoxybenzylidene acetal, acyclic acetals, such as tetrahydropyranyl (Thp), methoxymethyl (MOM), methoxyethoxymethyl (MEM), benzyloxymethyl (BOM) or methylthiomethyl (MTM). Particularly preferred. hydroxyl protecting groups are benzyl, acetyl, tert-butyl or TBS.

For the protecting group which is in each case employed, the literature discloses how to liberate the compounds of the formula I from their functional derivatives (e.g. T. W. Greene, P. G. M. Wuts, Protective Groups in Organic Chemistry, 2nd ed., Wiley, New York 1991 or P. J. Kocienski, Protecting Groups, 1 st ed., Georg Thieme Verlag, Stuttgart—New York, 1994). In this connection, it is also possible to use variants which are known per se but which are not mentioned here in more detail.

For example the BOC and O-tert-butyl groups can preferably be eliminated using TFA in dichloromethane or using approximately 3 to 5N HCl in dioxane at 15-30° C., while the Fmoc group is eliminated using an approximately 5 to 50% solution of dimethylamine, diethylamine or piperidine in DMF at 15-30° C. The Aloc group can be cleaved under mild conditions using precious metal catalysis in chloroform at 20-30° C. A preferred catalyst is tetrakis(triphenylphosphine)palladium(0).

As a rule, the starting compounds of formulae II to V are known. If they are novel, they can, nevertheless, be prepared using known methods which are known per se.

Compounds of the formula II are obtained, for example, from coupling the corresponding 2-aminopyridine derivatives with the corresponding n-bromocarboxylic esters (Br—[CH₂]ₙ—COOSG¹, where SG¹ is a hydroxyl protecting group as previously described) in the presence of a base and subsequently eliminating the protecting group under standard conditions.

Compounds of the formula IV are obtained by means of a peptide-analogous coupling of the compounds of the formula II with a glycine derivative H₂N—CH₂—COOSG², where SG² is a hydroxyl protecting group as previously described, under standard conditions.

Compounds of the formula V (β-amino acids) can be prepared in analogy with Skinner et al., J. Org. Chem. 1960, 25, 1756. Reacting the corresponding aldehyde R³—CHO with malonic acid and ammonium acetate in a suitable solvent, with alcohols, such as ethanol, being particularly preferred, generates the β-amino acid of the formula V, where R⁷ is H. Esterifying this free acid of the formula V under standard conditions yields compounds of the formula V where R⁷ is A.

In order to prepare the compounds of the formula I, the β-amino acids of the formula V which are protected on the acid function (either the compound is protected by means of an appropriate protecting group or R⁷ is A) are coupled to a glycine derivative SG³—NH—CH₂—COOH. The substituent SG³ of the glycine derivative SG³—NH—CH₂—COOH is an amino protecting group, as previously described, which is subsequently eliminated. Customary methods of peptide synthesis are described, for example, in Houben-Weyl, 1.c., volume 15/II, 1974, pages 1 to 806.

Compounds of the formula I can be obtained by reacting a compound of the formula II with a compound of the formula III and subsequently eliminating a protecting group or converting the radical R⁷, which denotes A, into the radical R⁷=H.

The compounds of the formula I can likewise be obtained by reacting a compound of the formula IV with a compound of the formula V and subsequently eliminating a protecting group or converting the radical R⁷, which denotes A, into the radical R⁷=H.

The coupling reaction is preferably effected in the presence of a dehydrating agent, for example of a carbodiimide such as dicyclohexylcarbodiimide (DCC), N-(3-dimethylaminopropyl)-N′-ethylcarbodiimide hydrochloride (EDC) or diisopropylcarbodiimide (DIC), or else, for example, propanephosphonic acid anhydride (cf. Angew. Chem. 1980, 92, 129), diphenylphosphoryl azide or 2-ethoxy-N-ethoxycarbonyl-1,2-dihydroquinoline, in an inert solvent, e.g., a halogenated hydrocarbon, such as dichloromethane, an ether, such as tetrahydrofuran or dioxane, an amide, such as DMF or dimethylacetamide, a nitrile, such as acetonitrile, in dimethyl sulfoxide or in the presence of these solvents, at temperatures of between about −10 and 40° C., preferably between 0 and 30° C. Depending on the conditions employed, the reaction time is between a few minutes and several days.

Addition of the coupling reagent TBTU (O-(benzotriazol-1-yl)-N,N,N′,N′-tetramethyluronium tetrafluoroborate) or O-(benzotriazol-1-yl)-N,N,N′,N′-tetramethyluronium hexafluorophosphate has proved to be particularly advantageous since only a small degree of racemization occurs in the presence of one of these compounds and no cytotoxic by-products are formed.

Instead of compounds of the formulae II and/or IV, it is also possible to use derivatives of compounds of the formulae II and/or IV, preferably a preactivated carboxylic acid, or a carbonyl halide, a symmetrical or mixed anhydride or an active ester. Such radicals for activating the carboxyl group in typical acylation reactions are described in the literature (e.g. in the standard works such as Houben-Weyl, Methoden der organischen Chemie [Methods of organic chemistry], Georg-Thieme-Verlag, Stuttgart). Activated esters are expediently formed in situ, for example by adding HOBt (1-hydroxybenzotriazole) or N-hydroxysuccinimide.

As a rule, the reaction takes place in an inert solvent; when a carbonyl halide is used, it takes place in the presence of an acid-binding agent, preferably an organic base such as triethylamine, dimethylaniline, pyridine or quinoline.

The addition of an alkali metal or alkaline earth metal hydroxide, carbonate or bicarbonate, or of another salt of a weak acid with alkali metals or alkaline earth metals, preferably potassium, sodium, calcium or cesium, can also be advantageous.

A base of the formula I can be converted with an acid into the pertinent acid addition salt, for example by reacting equivalent quantities of the base and the acid in an inert solvent such as ethanol and then concentrating by evaporation. Acids which yield physiologically harmless salts are particularly suitable for this reaction. Thus, it is possible to use inorganic acids, for example sulfuric acid, sulfurous acid, hexaoxodisulfuric acid, nitric acid, hydrohalic acids, such as hydrochloric acid or hydrobromic acid, phopshoric acids, such as orthophoshoric acid, or sulfamic acid, and also organic acids, in particular aliphatic, alicyclic, araliphatic, aromatic or heterocyclic monobasic or polybasic carboxylic sulfonic or sulfuric acids, for example formic acid, acetic acid, propionic acid, hexanoic acid, octanoic acid, decanoic acid, hexadecanoic acid, octadecanoic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methanesulfonic acid or ethanesulfonic acid, benzenesulfonic acid, trimethoxybenozic acid, adamantanecarboxylic acid, p-toluenesulfonic acid, glycolic acid, embonic acid, chlorophenoxyacetic acid, aspartic acid, glutamic acid, proline, glyoxylic acid, palmitic acid, parachlorophenoxyisobutyric acid, cyclohexanecarboxylic acid, glucose 1-phosphate, naphthalenemonosulfonic and naphthalenedisulfonic acids or lauryl sulfuric acid. Salts with acids which are not physiologically harmless, e.g. picrates, can be used for isolating and/or purifying the compounds of the formula I. On the other hand, the compounds of the formula I can be converted with bases (e.g. sodium or potassium hydroxide or carbonate) into the corresponding metal salts, in particular alkali metal salts or alkaline earth metal salts, or into the corresponding ammonium salts.

The invention also relates to compounds of the formula I, and their physiologically harmless salts or solvates, as drug active compounds.

The invention furthermore relates to compounds of the formula I, and their physiologically harmless salts or solvates, as integrin inhibitors.

The invention also relates to the compounds of the formula I, and their physiologically harmless salts or solvates, for use in controlling diseases.

The invention furthermore relates to pharmaceutical preparations which comprise at least one compound of the formula I, and/or one of its physiologically harmless salts or solvates, which is/are, in particular, prepared by a nonchemical route. In this connection, the compounds of the formula I can be brought into a suitable dosage form together with at least one solid, liquid and/or semiliquid excipient or adjuvant and, where appropriate, in combination with one or more additional active compounds.

These preparations can be used as drugs in human or veterinary medicine. Suitable excipients are organic or inorganic substances which are suitable for enteral (e.g. oral), parenteral or topical administration and which do not react with the novel compounds, for example water, vegetable oils, benzyl alcohols, alkylene glycols, polyethylene glycols, glyceryl triacetate, gelatin, carbohydrates, such as lactose or starch, magnesium stearate, talc and vaseline. Tablets, pills, coated tablets, capsules, powders, granules, syrups, juices and drops are used, in particular, for oral applications, while suppositories are used for rectal applications, solutions, preferably oily or aqueous solutions, and, in addition, suspensions, emulsions and implants are used for parenteral applications and ointments, creams or powders are used for topical applications. The novel compounds can also be lyophilized and the resulting lyophilisates used, for example, for producing preparations for injection. The abovementioned preparations can be sterilized and/or comprise adjuvants such as glidants, preservatives, stabilizers, wetting agents, emulsifiers, salts for influencing the osmotic pressure, buffering substances, dyes, flavorants and/or several additional active compounds, e.g. one or more vitamins.

For administration as an inhalation spray, it is possible to use sprays which comprise the active compound either dissolved or suspended in a propellant gas or propellant gas mixture (e.g. CO₂ or fluorochlorinated hydrocarbons). In this connection, the active compound is expediently used in micronized form, with it being possible for one or more additional physiologically tolerated solvents, e.g. ethanol, to be present. Inhalation solutions can be administered using customary inhalers.

The compounds of the formula I, and their physiologically harmless salts or solvates, can be used as integrin inhibitors in controlling diseases, in particular thromboses, cardiac infarction, coronary heart diseases, arteriosclerosis, tumors, osteoporosis, inflammations and infections.

The compounds of the formula I and/or their physiologically harmless salts are also used in connection with pathological processes which are maintained or propagated by angiogenesis, in particular in connection with tumors and rheumatoid arthritis.

In this connection, the substances according to the invention are as a rule administered in analogy with the compounds described in WO 97/26250 or WO 97/24124, preferably in doses of between about 0.05 and 500 mg, in particular of between 0.5 to 100 mg, per dosage unit. The daily dose is preferably between about 0.01 and 2 mg/kg of body weight. However, the specific dose for each patient depends on a very wide variety of factors, for example on the efficacy of the specific compound employed, on the age, on the body weight, on the general state of health, on the sex, on the diet, on the time and route of administration, on the rate of excretion, on the drug combination and on the severity of the particular disease to which the therapy applies. Parenteral administration is preferred.

Furthermore, the compounds of the formula I can be used as integrin ligands for preparing columns for affinity chromatography for the purpose of purifying integrins.

In this connection, the ligand, i.e. a compound of the formula I, is covalently coupled to a polymeric support by way of an anchoring function, for example the carboxyl group.

Suitable polymeric support materials are the polymeric solid phases which are known per se in peptide chemistry and which preferably exhibit hydrophilic properties, for example crosslinked polymeric sugars such as cellulose, Sepharose or Sephadex_{R}, acrylamides, polyethyleneglycol-based polymers or Tentakel polymers_{R}.

The materials for the affinity chromatography for purifying integrins are prepared under conditions which are customary for condensing amino acids and which are known per se.

The compounds of the formula I contain one or more chiral centers and can therefore be present in racemic form or in optically active form. Racemates which are obtained can be mechanically or chemically separated into the enantiomers using methods which are known per se. Preference is given to forming diastereomers from the racemic mixture by reacting it with an optically active separating agent. Examples of suitable separating agents are optically active acids, such as the D and L forms of tartaric acid, diacetyltartaric acid, dibenzoyltartaric acid, mandelic acid, malic acid, lactic acid or various optically active camphorsulfonic acids, such as β-camphorsulfonic acid. It is also advantageous to perform an enantiomer separation using a column which is filled with an optically active separating agent (e.g. dinitrobenzoylphenylglycine); an example of a suitable eluent is a hexane/isopropanol/acetonitrile mixture, for example in the volume ratio 82:15:3.

It is naturally also possible to obtain optically active compounds of the formula I by means of the above-described methods by using starting compounds which are already optically active.

The invention is explained in more detail by means of the examples which follow.

Both in the above text and in that which follows, all the temperatures are given in ° C. In the examples, “customary working up” denotes the following: water is added, if necessary, the pH is adjusted, if necessary and depending on the constitution of the end product, to values of between 2 and 10, the mixture is extracted with ethyl acetate or dichloromethane, the phases are separated, the organic phase is dried over sodium sulfate and concentrated by evaporation, and the residue is purified by chromatography on silica gel, by preparative HPLC and/or by crystallization. The purified compounds are freeze-dried, where appropriate.

RT=retention time (in minutes) in HPLC in the following systems:

Column: Lichrosorb RP-18 (5 μm) 250×4 mm; (analytical) Lichrosorb RP-18 (15 μm) 250×50 mm; (preparative)

Analytical HPLC

The eluents employed are gradients composed of (A) 0.1% TFA and (B) 0.1% TFA in 9 parts of acetonitrile and 1 part of water. The gradient is given in percent by volume of acetonitrile. The gradient runs for 5 min at 20% B and then for 50 min at 90% B. The retention times which are obtained with regard to the Lichrosorb RP-18 (5 μm) 250×4 mm column are given in min. In the case of very polar substances, another gradient is used: 5 min at 5% B and then 50 min at 75% B. The retention times in this gradient are indicated by *. Detection is effected at 225 nm.

Preparative HPLC:

The Lichrosorb RP-18 (15 μm) 250×50 mm column is used. The individual fractions are examined analytically and pooled. The substances are then freeze-dried. The compounds which are purified by means of preparative HPLC are isolated as trifluoroacetates.

Molecular weights are determined by means of mass spectrometry (MS) using FAB (fast atom bombardment): designated “MS-FAB (M+H)⁺” in that which follows.

Abbreviations:

TBTU: (2-(1H-benzotriazol-1-yl)-1,1,3,3-tetramethyluronium tetrafluoroborate)

HOBt: (1-hydroxybenzotriazole)

### EXAMPLE 1

Preparing 4-quinolin-8-ylbenzaldehyde:

1 g of 8-bromoquinoline, 720 mg of 4-formylphenylboronic acid and 166 mg of tetrakis(triphenylphosphine)palladium(0) are dissolved in toluene, and an aqueous solution of sodium carbonate (1 g in 3 ml) is added to this solution. The reaction mixture is boiled under reflux overnight.

The organic phase was separated off and washed 2× with water. The organic phase was then dried with anhydrous sodium sulfate and filtered and the solvent was stripped off on a rotary evaporator. For purificatio, a silica gel chromatograph is carried out in dichloromethane/methanol 9/1. The fractions containing the product are triturated with diethyl ether. 450 mg of pale pink crystals are obtained. TLC in chloroform/methanol/glacial acetic acid 85/10/5: Rf=0.75

### EXAMPLE 2

Preparing the β-amino acids:

The β-amino acid is prepared as described in the cited references. 110 mg of the β-amino acid 3-amino-3-(4-quinolin-8-ylphenyl)propionic acid are obtained from 450 mg of 4-quinolin-8-ylbenzaldehyde, 200 mg of malonic acid and 300 mg of ammonium acetate.

The other β-amino acids are prepared in an analogous manner. For the subsequent reaction, an ethyl ester is prepared by the customary method (boiling with thionyl chloride/ethanol).

References:

Skinner et al.; J. Org. Chem. 25, 1756 (1960)

E. Profft, F.-J. Becker; Journal fur Praktische Chemie [Journal of Practical Chemistry], 30, 18-38 (1965)

### EXAMPLE 3

### Synthesizing 3-{2-[4-(4-methylpyridin-2-ylamino)butanoylamino]ethanoylamino}-3-(4-quinolin-8-ylphenyl)propionic acid

30 mg of ethyl 3-amino-3-(4-quinolin-8-ylphenyl)propionate hydrochloride are dissolved in 5 ml of DMF, and 63 mg of [4-(4-methylpyridin-2-ylamino)butanoylamino]acetic acid are added to this solution. The whole is cooled down to approx. 0° C. and 30 mg of TBTU and 4.3 mg of HOBt are added at this temperature. The mixture is neutralized with approx. 0.4 ml of N-methylmorpholine. It is then stirred overnight at room temperature.

The clear solution was stripped down to the residue. 30 ml of ethyl acetate were then added to the residue and the whole was extracted twice with 20 ml of half-concentrated bicarbonate solution and washed once again with 30 ml of saturated sodium chloride solution. The organic phase is dried over sodium sulfate, filtered and concentrated.

The resulting crude material of 50 mg in weight is dissolved in 4.5 ml of ethanol and 0.5 ml of NaOH (2 mol/l) is added. The mixture is stirred overnight at room temperature. The solution is evaporated down on a rotary evaporator and the residue is purified by means of preparative HPLC. 22 mg of the abovementioned substance are obtained.

FAB MS (M+H⁺) 526; retention time (RT) 13.88 min

### EXAMPLE 4

The following compounds were prepared in a similar manner to that described in Examples 1 to 3.

| | |
|---|---|
| 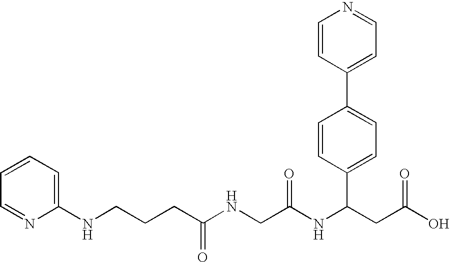 | a) 3-{2-[4-(4-Pyridin- 2-ylamino)butanoylamino]- ethanoylamino}-3-(4-pyridin-4-yl- phenyl)propionic acid FAB MS [M + H⁺]: 462 |
| 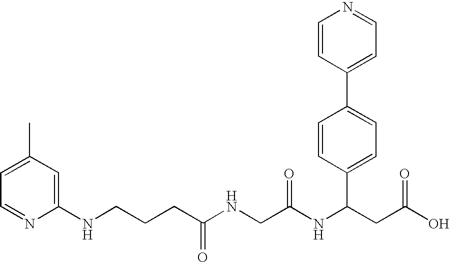 | b) 3-{2-[4-(4-Methylpyridin-2- ylamino)butanoylamino]- ethanoylamino}-3-(4-pyridin-4- ylphenyl)propionic acid FAB MS [M + H⁺]: 476 |
| 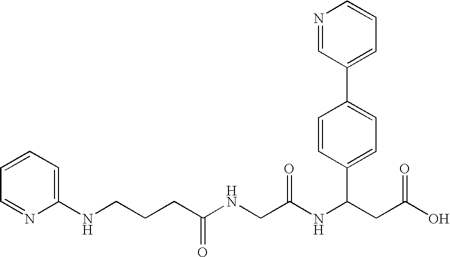 | c) 3-{2-[4-(Pyridin-2- ylamino)butanoylamino]- ethanoylamino}-3-(4-pyridin-3-yl- phenyl)propionic acid FAB MS [M + H⁺]: 462 |
| 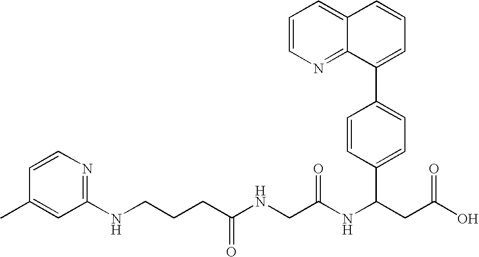 | d) 3-{2-[4-(4-Methylpyridin-2- ylamino)butanoylamino]- ethanoylamino}-3-(4-quinolin-8- ylphenyl)propionic acid FAB MS [M + H⁺]: 526 RT: 13.88 min |
| 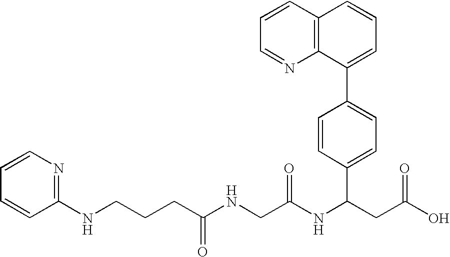 | e) 3-{2-[4-(Pyridin- 2-ylamino)butanoylamino]- ethanoylamino}-3-(4-quinolin-8-yl- phenyl)propionic acid FAB MS [M + H⁺]: 512 RT: 10.33 min |
| 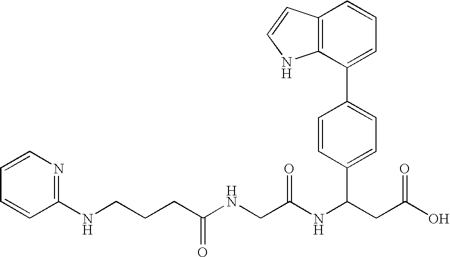 | f) 3-[4-(1H-Indol-7-yl)-phenyl]- 3-{2-[4-(pyridin-2- ylamino)butanoyl- amino]ethanoylamino}propionic acid FAB MS [M + H⁺]: 500 RT: 25.81 min |
| 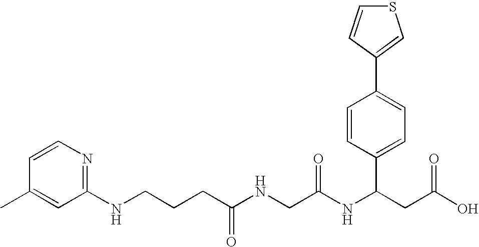 | g) 3-{2-[4-(4-Methylpyridin-2- ylamino)butanoylamino]- ethanoylamino}-3-(4-thiophen-3- ylphenyl)propionic acid FAB MS [M + H⁺]: 481 RT: 23.92 min |
| 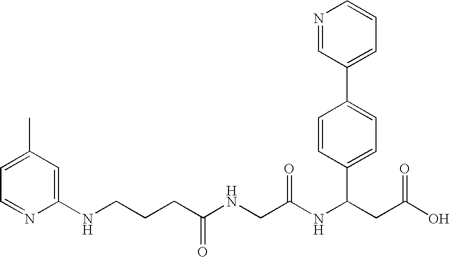 | h) 3-{2-[4-(Pyridin- 2-ylamino)butanoylamino]- ethanoylamino}-3-(4-thiophen-3-yl- phenyl)propionic acid FAB MS [M + H⁺]: 467 RT: 22.32 min |
| 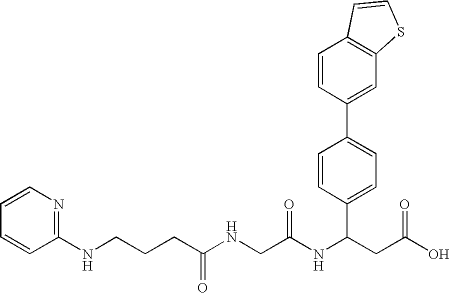 | i) 3-{2-[4-(4-Methylpyridin-2- ylamino)butanoylamino]- ethanoylamino}-3-(4-pyridin-3- ylphenyl)propionic acid FAB MS [M + H⁺]: 476 |
| 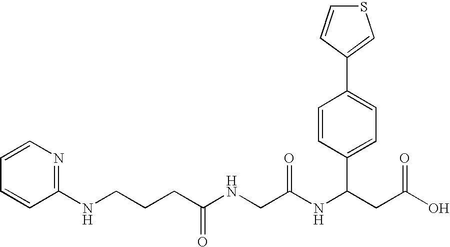 | j) 3-(4-Benzo[b!]thiophen-6- ylphenyl)-3-(2-[4-(pyridin-2- ylamino)butanoylamino]ethanoyl- amino}propionic acid FAB MS [M + H⁺]: 517 |

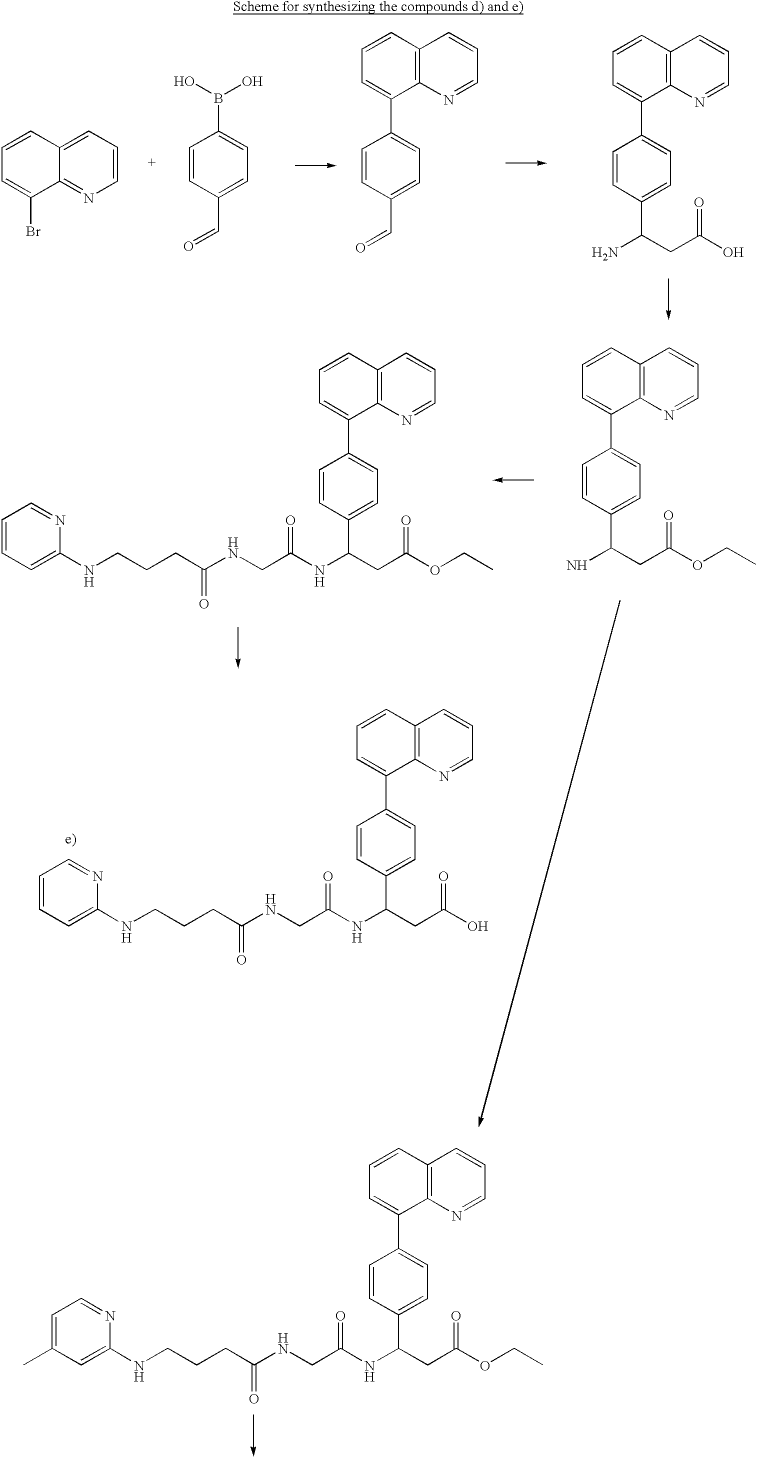

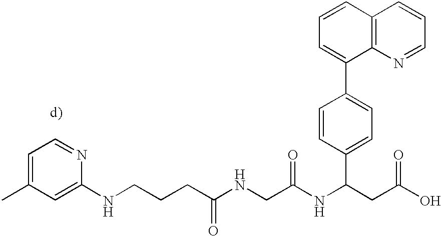

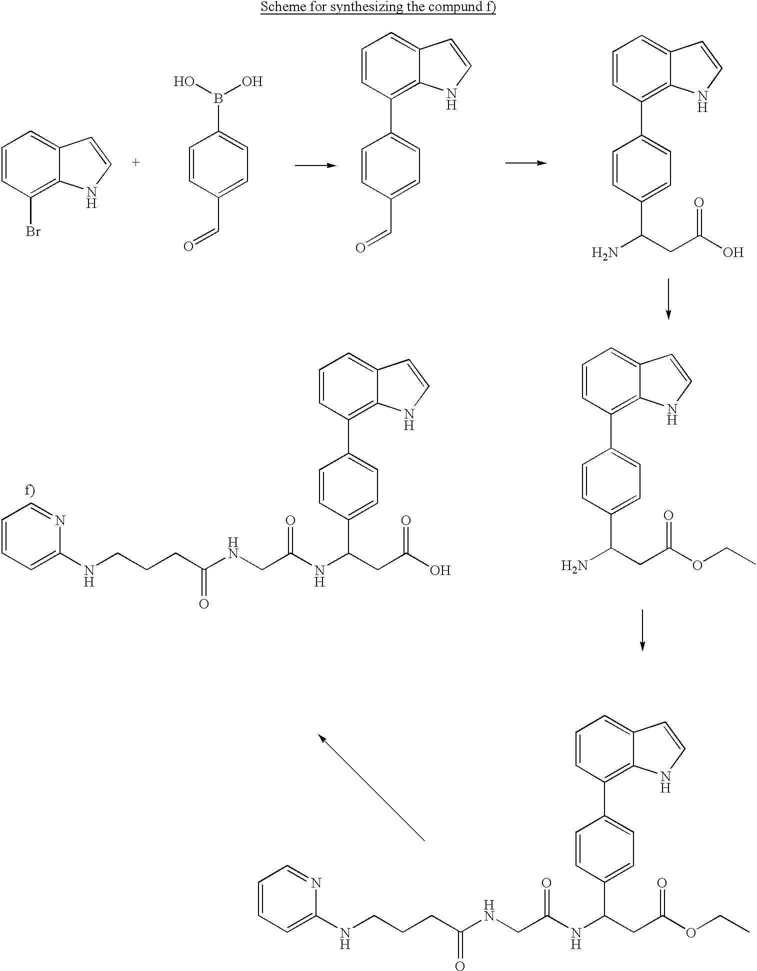

### EXAMPLE 5

Assays

The angiogenic blood vessels of a tumor display the αvβ3 integrin conspicuously and can in this way be tracked down deliberately using αvβ3-specific inhibitors.

Using an analytical method, it was possible to identify cell lines which could be isolated form human tumors and which present the integrin αvβ6 but not the integrin αvβ3 for example Detroit 562, HT-29 and UCLA-P3, or which present the two integrins αvβ3 and αvβ6, for example Calu-3 and Capan-2. (Analytical methods: immunoprecipitation and fluorescence-activated cell sorting analysis). These identified cell lines grow as subcutaneous tumors in immunodeficient rodents, e.g. in nu/nu mice.

As has already been described, inhibitors of the αvβ3 integrin receptor block tumor growth by the blood vessels which are growing into the tumor being exposed to apoptotic signals and dying as a result of programmed cell death (apoptosis). (References: P. C. Brooks, Eur. J. Cancer 1996, 32A, 2423-2426, P. C. Brooks et al., Cell 1994, 79, 1157-1164 or S. Stomblad et al., J. Clin. Invest 1996, 98, 426-433.)

The αvβ6 integrin inhibitors impair tumor development directly. The synergistic effect of the combined therapy in accordance with the invention is demonstrated by the following series of experiments, which were carried out in analogy with the test systems described by Mitjans et al., J. Cell. Sci. 1995, 108, 2825-2838: αvβ6-expressing tumor cells are implanted subcutaneously, for example in nu/nu mice. In analogy with the M21 cell line described by Mitjans et al., the effect of the integrin inhibitors on the growth of these tumor cells in the mice is then observed.

After the tumor cells have been implanted, the mice which have been prepared in this way are separated and divided up into groups of 10 mice each. The mice are treated daily in accordance with the invention, by being injected intraperitoneally with the appropriate integrin inhibitors, and the growth of the tumors is observed. The control group is given in injections of sterile, pyrogen-free saline solution. The tumor size is measured twice a week and the corresponding tumor volume is calculated.

The following examples relate to pharmaceutical preparations:

### Example A

Injection Vials

A solution of 100 g of an active compound of the formula I and 5 g of disodium hydrogen phosphate in 3 l of double distilled water is adjusted to pH 6.5 with 2N hydrochloric acid, sterilized by filtration, aliquoted into injection vials and lyophilized under sterile conditions; the vials are then sealed under sterile conditions. Each injection vial contains 5 mg of active compound.

### Example B

Suppositories

A mixture consisting of 20 g of an active compound of the formula I, 100 g of soybean lecithin and 1400 g of cocoa butter is melted and poured into molds and allowed to cool. Each suppository contains 20 mg of active compound.

### Example C

Solution

A solution consisting of 1 g of an active compound of the formula I, 9.38 g of NaH₂PO₄.2H₂O, 28.48 g of Na₂HPO₄.12H₂O and 0.1 g of benzalkonium chloride in 940 ml of double distilled water is prepared. It is adjusted to pH 6.8, made up to 1 l and sterilized by irradiation. This solution can be used in the form of eye drops.

### Example D

Ointment

500 mg of an active compound of the formula I are mixed with 99.5 g of vaseline under aseptic conditions.

### Example E

Tablets

A mixture of 1 kg of active compound of the formula I, 4 kg of lactose, 1.2 kg of potato starch, 0.2 kg of talc and 0.1 kg of magnesium stearate is pressed into tablets in the customary manner such that each tablet comprises 10 mg of active compound.

### Example F

Coated Tablets

Tablets are pressed in analogy with Example E, with these tablets then being covered, in a customary manner, with a coating composed of sucrose, potato starch, talc, tragacanth and colorant.

### Example G

Capsules

2 kg of active compound of the formula I are aliquoted, in the customary manner, into hard gelatin capsules such that each capsule contains 20 mg of the active compound.

### Example H

Ampoules

A solution of 1 kg of active compound of the formula I in 60 l of double distilled water is sterilized by filtration, aliquoted into ampoules and lyophilized under sterile conditions; the ampoules are then sealed under sterile conditions. Each ampoule contains 10 mg of active compound.

### Example I

Inhalation Spray

14 g of active compound of the formula I are dissolved in 10 l of an isotonic solution of NaCl and the resulting solution is aliquoted into commercially available spraying vessels having a pump mechanism. The solution can be sprayed into the mouth or the nose. One puff (about 0.1 ml) corresponds to a dose of about 0.14 mg.

1. A compound of the formula I 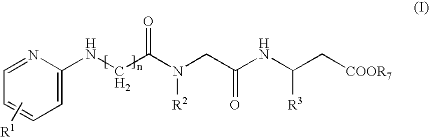
where
R¹ is H, A, Ar, Hal, —OH, —O—A, —CF₃ or —OCF₃;
R² and R⁷ are H or A;
R³ is 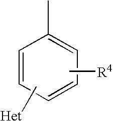
R4, R5 and R6 are in each case, independently of each other, H, A, Hal, —OH, —O—A, —CF3, —OCF3, —CN, —NH2, —A—NH2;
A is C1-C6-alkyl;
Ar is a substituent which is formed by an aromatic radical which is optionally substituted once, twice or three times by R⁵ and which has from 1 to 3 ring structures which are optionally fused with other ring structures to form a fused ring system;
Het is a substituent which is formed by a heterocycle which has from 1 to 3 ring structures, with each ring structure being saturated, unsaturated or aromatic and being optionally fused with other ring structures to form a fused ring system, and the heterocycle possessing a total of from 1 to 4 N, O and/or S atoms in the ring structures and being optionally substituted by R⁶;
Hal is F, Cl, Br or I;
n is 2, 3, 4, 5 or 6
and the well-tolerated salts and solvates thereof.

2. A compound as claimed in claim 1, selected from
a) 3-{2-[4-(4-Pyridin-2-ylamino)butanoylamino]ethanoylamino}-3-(4-pyridin-4-ylphenyl)propionic acid
b) 3-{2-[4-(4-Methylpyridin-2-ylamino)butanoylamino]ethanoylamino}-3-(4-pyridin-4-ylphenyl)propionic acid
c) 3-{2-[4-(Pyridin-2-ylamino)butanoylamino]ethanoylamino}-3-(4-pyridin-3-ylphenyl)propionic acid
d) 3-{2-[4-(4-Methylpyridin-2-ylamino)butanoylamino]ethanoylamino}-3-(4-quinolin-8-ylphenyl)propionic acid
e) 3-{2-[4-(Pyridin-2-ylamino)butanoylamino]ethanoylamino}-3-(4-quinolin-8-ylphenyl)propionic acid
f) 3-[4-(1H-Indol-7-yl)-phenyl]-3-{2-[4-(pyridin-2-ylamino)butanoylamino]ethanoylamino}propionic acid
g): 3-{2-[4-(4-Methylpyridin-2-ylamino)butanoylamino]ethanoylamino}-3-(4-thiophen-3-ylphenyl)propionic acid
h) 3-{2-[4-(Pyridin-2-ylamino)butanoylamino]ethanoylamino}-3-(4-thiophen-3-ylphenyl)propionic acid
i) 3-{2-[4-(4-Methylpyridin-2-ylamino)butanoylamino]ethanoylamino}-3-(4-pyridin-3-yl-3-(4-benzo[b!]thiophen-6-ylphenyl)-3-{2-[4-(pyridin-2-ylamino)butanoylamino]ethanoylamino}propionic acid
and the well-tolerated salts and solvates thereof.

3. A process for preparing the compounds of the formula I as claimed in claim 1 or claim 2, and their salts and solvates, wherein
(a) a compound of the formula II 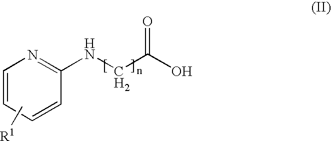
where R¹ and n have the meanings given in claim 1, is reacted with a compound of the formula III 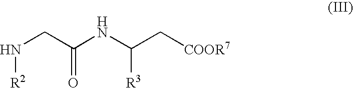
where R², R³ and R⁷ have the meanings given in claim 1, and the radical R⁶≠H is optionally converted into the radical R⁶=H, where appropriate, or
(b) a compound of the formula IV 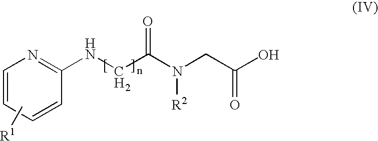
where R¹, R² and n have the meanings given in claim 1, is reacted with a compound of the formula V 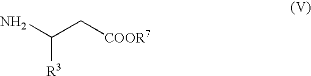
where R³ and R⁷ have the meanings given in claim 1, and the radical R⁷≠H is converted into the radical R⁷=H, where appropriate, or
(c) in a compound of the formula I, one or more of the radicals R¹, R², R³, R⁴, R⁵, R⁶ and/or R⁷ is/are converted into one or more of the radicals R¹, R², R³, R⁴, R⁵, R⁶ and/or R⁷ by
vii) a hydroxyl group being alkylated and/or
viii) an ester group being hydrolyzed to a carboxyl group and/or
ix) a carboxyl group being esterified and/or
x) an amino group being alkylated and/or
xi) an amino group being acylated and/or
xii) a basic or acidic compound of the formula I being converted into one of its salts or solvates by being treated with an acid or base.

4. A drug which comprises compounds as claimed in claim 1 or claim 2.

5. A pharmaceutical preparation, which has a content of at least one compound as claimed in claim 1 or claim 2.

6. The use of compounds as claimed in claim 1 or claim 2 for producing a drug.

7. The use of the compounds as claimed in claim 1 or claim 2 as integrin inhibitors.

8. The use of the compounds claimed in claim 1 or claim 2 as inhibitors of the integrins αvβ1, αvβ3, αvβ5, αvβ6 and αllbβ3.

9. The use of the compounds as claimed in claim 1 or claim 2 as inhibitors of the integrins αvβ3, αvβ5 and αvβ6.

10. The use of compounds as claimed in claim 1 or claim 2 for the therapy of pathological processes which are maintained or propagated by angiogenesis.

11. The use of compounds as claimed in claim 1 or claim 2 for producing a drug for controlling thromboses, cardiac infarction, coronary heart disease, arteriosclerosis, inflammations, tumors, osteoporosis, infections and restenosis following angioplasty.
